SEQUENCE LISTING (15 sequences)

SEQ ID NO: 1 - protein, 101 aa, Homo sapiens
MSSLPVPYKLPVSLSVGSCVIIKGTPIHSFINDPQLQVDFYTDMDEDSDIAFRFRVHFGNHVVMNRREFGIWMWRRQQTTCPLRMANNLSCASTYITMSMR

SEQ ID NO: 2 - protein, 109 aa, Homo sapiens
MSSLPVQVDFYTDMDEDSDIAFRFRVHFGNHVVMNRREFGIWMLEETTDYVPFEDGKQFELCIYVHYNEYEIKVNGIRIYGFVHRIPPSFVKMVQVSRDISLTSVCVCN

SEQ ID NO: 3 - DNA, 500 bp, Homo sapiens
agaagactggactcaattctgaaggtcgccaagaaggagagaacaatgtcttctttacccgtgccatacaaactgcctgtgtctttgtctgttggttcctgcgtgataatcaaagggacaccaatccactcttttatcaatgacccacagctgcaggtggatttctacactgacatggatgaggattcagatattgccttccgtttccgagtgcactttggcaatcatgtggtcatgaacaggcgtgagtttgggatatggatgttggaggagacaacagactacgtgccctttgaggatggcaaacaatttgagctgtgcatctacgtacattacaatgagtatgagataaaggtcaatggcatacgcatttacggctttgtccatcgaatcccgccatcatttgtgaagatggtgcaagtgtcgagagatatctccctgacctcagtgtgtgtctgcaattgagggagatgatcacactcctcattgttgaggaatcc

SEQ ID NO: 4 - DNA, 500 bp, Homo sapiens
tcttctgacctgagttaagacttccagcggttcttcctctcttgttacagaagaaatgggcacggtatgtttgacggacacagaaacagacaaccaaggacgcactattagtttccctgtggttaggtgagaaaatagttactgggtgtcgacgtccacctaaagatgtgactgtacctactcctaagtctataacggaaggcaaaggctcacgtgaaaccgttagtacaccagtacttgtccgcactcaaaccctatacctacaacctcctctgttgtctgatgcacgggaaactcctaccgtttgttaaactcgacacgtagatgcatgtaatgttactcatactctatttccagttaccgtatgcgtaaatgccgaaacaggtagcttagggcggtagtaaacacttctaccacgttcacagctctctatagagggactggagtcacacacagacgttaactccctctactagtgtgaggagtaacaactccttagg

SEQ ID NO: 5 - protein, 15 aa, Homo sapiens
RRLDSILKVAKKERT

SEQ ID NO: 6 - protein, 139 aa, Homo sapiens
MSSLPVPYKLPVSLSVGSCVIIKGTPIHSFINDPQLQVDFYTDMDEDSDIAFRFRVHFGNHVVMNRREFGIWMLEETTDYVPFEDGKQFELCIYVHYNEYEIKVNGIRIYGFVHRIPPSFVKMVQVSRDISLTSVCVCN

SEQ ID NO: 7 - protein, 2 aa, Homo sapiens
GR

SEQ ID NO: 8 - protein, 9 aa, Homo sapiens
SNSSLLRNP

SEQ ID NO: 9 - DNA, 329 bp, Homo sapiens
atgtcttctttacccgtgcaggtggatttctacactgacatggatgaggattcagatattgccttccgtttccgagtgcacttcggcaatcatgtggtcatgaacaggcgtgagtttgggatatggatgttggaggagacaacagactacgtgccctttgaggatggcaaacaatttgagctgtgcatctgcgtacattacaatgagtatgagataaaggtcaatggcatacgcatttacggctttgtccatcgaatcccgccatcatttgtgaagatggtgcaagtgtcgagagatatctccctgacctcagtgtgtgtctgcaatga

SEQ ID NO: 10 - DNA, 329 bp, Homo sapiens
tacagaagaaatgggcacgtccacctaaagatgtgactgtacctactcctaagtctataacggaaggcaaaggctcacgtgaagccgttagtacaccagtacttgtccgcactcaaaccctatacctacaacctcctctgttgtctgatgcacgggaaactcctaccgtttgttaaactcgacacgtagacgcatgtaatgttactcatactctatttccagttaccgtatgcgtaaatgccgaaacaggtagcttagggcggtagtaaacacttctaccacgttcacagctctctatagagggactggagtcacacacagacgttact

SEQ ID NO: 11 - DNA, 306 bp, Homo sapiens
atgtcttctttacccgtgccatacaaactgcctgtgtctttgtctgttggttcctgcgtgataatcaaagggacaccaatccactcttttatcaatgacccacagctgcaggtggatttctacactgacatggatgaggattcagatattgccttccgtttccgagtgcactttggcaatcatgtggtcatgaacaggcgtgagtttgggatatggatgtggaggagacaacagactacgtgccctttgaggatggcaaacaatttgagctgtgcatctacgtacattacaatgagtatgagataa

SEQ ID NO: 12 - DNA, 26 bp, Artificial sequence
  sense primer
cgaatccatgtcttctttacccgtgc

SEQ ID NO: 13 - DNA, 32 bp, Artificial sequence
  anti-sense primer
taagtcgagctccatccatatcccaaactcac

SEQ ID NO: 14 - DNA, 31 bp, Artificial sequence
  sense primer
cgatacggatccatgtcttctttacccgtgc

SEQ ID NO: 15 - DNA, 32 bp, Artificial sequence
  anti-sense primer
taagtcgagctcattgcagacacacactgagg

(19) United States
(12) Patent Application Publication
(10) Pub. No.: US 2008/0187929 A1
(43) Pub. Date: Aug. 7, 2008

(54) Method for determining preeclampsia risk

(76) Inventors: Hamutal Meiri, Tel Aviv (IL); Marei Sammar, Tamra (IL); Renate Hillermann, Western Cape (ZA)

Correspondence Address: THE NATH LAW GROUP, 112 South West Street, Alexandria, VA, 22314, US

(21) Appl. No.: 11/979,549

(22) Filed: Nov. 5, 2007

Related U.S. Application Data
(60) Provisional application No. 60/898,707, filed on Feb. 1, 2007.

(51) Int. Cl. C12Q 1/02; C12Q 1/68; G01N 33/00; C07K 14/00; C12P 21/04; G01N 33/566; G01N 33/48

(52) U.S. Cl. 435/6; 436/94; 436/63; 435/29; 436/501; 530/350; 435/69.1

(57) ABSTRACT

A method for determining the risk of a mother of a fetus to develop preeclampsia during pregnancy comprising: (a) providing a sample of genomic DNA from an individual related to the fetus; (b) analyzing the DNA for the presence of one or more mutations in the PP13 gene; and (c) determining the risk of the mother on the basis of the presence of the mutations. Also disclosed are mutated PP13 protein variants, and a kit for use in the method of the invention comprising DNA probes for specific genomic sequences of the PP13 native gene and/or mutated sequences thereof.

This application claims the benefit of prior U.S. provisional patent application No. 60/898,707 filed Feb. 1, 2007, the contents of which are hereby incorporated by reference in their entirety.

### FIELD OF THE INVENTION

This invention relates to a screening method for identifying mutation variants of PP13 that can indicate the risk of developing preeclampsia.

### BACKGROUND OF THE INVENTION

The pregnancy disorder known as preeclampsia (PE) is an undesirable complication of pregnancy occurring in 5-7% of all pregnant women and it is the second most frequent cause of maternal death during pregnancy (18% of maternal mortality associated with pregnancy in the United States). The disorder is derived from placental insufficiency that results from impaired placentation and blood/oxygen/nutrient supply to the placenta that are followed by the clinical symptoms.

Preeclampsia is defined as a newly onset hypertension of ≧90/140 mm Hg (systolic/diastolic, at least one) measured on two occasions, 4-6 hours apart (and in some cases 4-72 hr apart) developed after 20 weeks of gestation in previously normotensive women in combination with proteinuria of ≧2+ in dipstick or >300 mg/dL in 24 hr urine collection determined in women with a previous history of no or trace protein in the urine.

Approximately fifty percent of all preeclampsia pregnancies are delivered via Cesarean section as compared with only 15-18% of pregnancies in the entire population. Women who experience preeclampsia disorders during pregnancy have a 9 times higher risk of consequently developing cardiovascular diseases thereby shortening their life expectancy. Although the proportion of pregnant women that develop PE is higher in developing countries, numbers in the USA remain high (5-7%).

Severe preeclampsia is defined as preeclampsia where the hypertension is ≧110/160 mm Hg (systolic/diastolic, at least one) and proteinuria of ≧3+ in dipstick or >3 gr/dL in 24 hr urine collection. In extreme situations, preeclampsia can turn into eclampsia, an emergency situation associated with convulsion, stroke, and coma that puts the mother at risk of losing life. As a result, women who develop severe preeclampsia are delivered within 48 hours to remove the placenta and save the mother's life.

Early preeclampsia is a form of severe preeclampsia defined as above where the severity of the disease requires delivery before term (<37 weeks of gestation). Often this form of the disease is accompanied by fetal restricted growth in the uterine (IUGR) that put not only the life of the mother but also the life of the fetus at risk. According to the NICHD, early preeclampsia accounts for 20-25% of all cases of PE, which means 1-2 out of 100 pregnant women of whom 15% are delivered before 34 weeks, i.e. the baby is born extremely premature, after experiencing a stressful pre-delivery period. The earlier the delivery occurs, the more severe are the complications to the baby, which combine baby low birth weight, incomplete internal organ maturation including blindness, motor and cognitive disorders and life-long medical disabilities, and increased risk to later develop hypertension, cardiovascular diseases and diabetes. The 10% of cases delivered before 34 gestational weeks (GW) are the most severe ones, responsible for most mortality cases, and the babies born, if they survive, need 6-8 weeks in neonatal intensive care units. According to the NICHD, this is the group for whom early detection is essential for its life saving capability and prevention of prematurity. The only current practice to treat preeclampsia is to deliver the mother, and when such a delivery takes place prematurely, a variety of impairments of the newborn baby appear due to lower birth weight, motor and cognitive disabilities, and, in very severe cases, in-partum or after partum death. Many studies have been carried out to try and identify at an early stage the risk of developing preeclampsia.

The disorder usually breaks out in the third trimester but the underlying pathological processes in the placenta develop very early. Thus early detection of a risk to develop preeclampsia either before conception or during pregnancy can provide a means to prevent the disease or ease the severity of the symptoms.

1) Early detection enables to manage the risk by a close surveillance of the woman. Effective increased surveillance for women at high risk is in compliance with the guidelines of the American College of Obstetric and Gynecology (“ACOG guidelines”), and the increased surveillance in high-risk cases significantly improves outcome. Pregnancy Management Programs were shown to save costs due to close surveillance coupled with education and awareness programs given to the participating pregnant women. Among benefits are a drop of births due to early PE (<34 weeks) only 0.6% of all deliveries compared with the national average of 1.96%, premature delivery reduction to 0.9% of all deliveries compared with the national benchmark of 2.3% and low birth weight due to preeclampsia being 1.3% compared with the national average of 2.9%. The close surveillance allows pregnant women to reach a tertiary level medical center before birth, an issue of great significance in community clinics and rural-based health service settings. The other benefit is to buy time to administer treatments and drugs such as antenatal corticosteroids that facilitate the maturation of fetal organs. The close surveillance enables in certain cases to extend pregnancy duration so as to reduce the severity of newborn pre-maturely.

2) The early detection enables a longer period for developing drug intervention strategies using various putative agents that are considered to be working on the placenta to prevent/reduce the risk. Although there is no gold standard for treatment, a number of candidates have shown promise, including low dose aspirin, low molecular weight heparin, anti oxidants such as vitamin C and E and magnesium sulfate, among others. In all of these studies not all women at risk benefited from the therapeutic intervention. While in some cases there are indications that the intervention was initiated too late, in other cases there is no clear evidence to indicate if the medication used wasn't the right one or wasn't used at the right time or dose. Current studies show that it is necessary to tailor drug intervention to each woman that is most suitable for her from a putative medications list available today (as well as medications that will become available in due course), and to continuously monitor the effectiveness of the treatment.

3) For patients who are pre-disposed for preeclampsia based on family history or previous pregnancy history, particularly those who lost babies as a result of preeclampsia, detection before pregnancy could be of prime importance to plan for pregnancy, and take preventive measures as explained above. For women who take part in an in-vitro fertilization program, detection of elevated prior risk of preeclampsia could be an advantage to manage the implantation and to select implants who will not be associated with elevated risk for preeclampsia

Placental Protein 13 (PP13) is a protein of 15-16,000 MW which may be purified from human placental tissue or prepared by recombinant technology as described in U.S. Pat. No. 6,548,306 (Admon, et al), the contents of which are incorporated herein by reference. The amino acid and DNA sequences of PP13 are shown in FIG. 5, and the amino acid sequence is shown in FIG. 8A. Purified PP13 was used to develop an assay for the detection of some pregnancy-related disorders such as intrauterine growth restriction (IUGR), preeclampsia and preterm delivery as described in U.S. Pat. No. 5,198,366 (Silberman), the contents of which are incorporated herein by reference. Both a radioimmunoassay (RIA) and an enzyme-linked immunosorbent assay (ELISA) were developed using labeled PP13 and anti PP13 polyclonal antiserum.

Analysis of the PP13 gene LGALS13 revealed its basic structure as comprising 3 introns and 4 exons that exhibit a high sequence homology to the galectin family—a group of proteins with high affinity to sugar residues which is particularly important in implantation (Than, N. G., et al (1999) Placenta 20:703-710; Than, et al., (2004) Eur. J Biochem. 271(6):1065-1078). Indeed PP13 was found by immunohistochemistry to be important in placentation.

U.S. Pat. No. 6,790,625, the contents of which are incorporated herein by reference, discloses monoclonal antibodies to PP13 and a solid-phase immunoassay capable of measuring maternal serum PP13 during the early stages of pregnancy. Indeed, Nicolaides et al., (2006) A novel approach to first-trimester screening for early pre-eclampsia combining serum PP-13 and Doppler ultrasound Ultrasound in Obstetrics and Gynecology, 27(1) pp. 13-17, have, as well as others, used the ELISA kit developed based on this patent to show at very high accuracy that lower PP13 in the first trimester corresponds to elevated risk for preeclampsia.

WO 04/021012, the contents of which are incorporated herein by reference, discloses a diagnostic method for pregnancy complications based on a number of factors, including PP13 level.

Stolk, M. et al. (2006) Hypertension in Pregnancy, Abstracts from the 15^{th} World Conference of the Intl. Soc. for the Study of Hyper. in Preg., Vol. 25 (Suppl. 1) P029, the contents of which are incorporated herein by reference, describes nucleotide sequence variations in the human galectin/placental protein 13 gene, LGALS13.

Sanmuar, M. et al. (2007) RNA splicing and DNA polymorphism leading to two shorter subforms of placenta protein 13 in preeclampsia, American College of Obstetric and Gynecology Conference, the contents of which are incorporated herein by reference, describes a spliced variant lacking exon-2 of the PP13 gene.

### SUMMARY OF THE INVENTION

It is an object of the present invention to provide a method for determining the risk of a pregnant woman to develop preeclampsia.

In one aspect of the present invention, there is provided a method for determining the risk of a mother of a fetus to develop preeclampsia during pregnancy comprising:
(a) providing a sample of genomic DNA containing the PP13 gene from an individual related to the fetus;
(b) analyzing the DNA for the presence of one or more mutations in the PP13 gene; and
(c) determining the risk of the mother on the basis of the presence of the mutations.

As is well known, the genotype of the placenta, which is the major source of PP13, is derived from that of the fetus. Thus, in order to determine whether a risk exists for the existence of a mutation in the PP13 gene in the placental genome, it is sufficient to determine the genotype of the parents or a sibling of the fetus or to perform haplotype construction and analysis, investigate transmission patterns, investigate transmission patterns (mother-to-baby), identify evidence of imprinting, and other genetic analysis methods. Thus, the individual related to the fetus in the method of the invention may be selected from one or more of the following:
(a) the mother of the fetus, or her mother or father;
(b) the father of the fetus, or his mother or father;
(c) a sibling of the fetus or siblings of the father or of the mother of the fetus (although a negative result is not conclusive);
(d) the placenta of the pregnancy or of a twin pregnancy or the placenta from previous pregnancies of the mother; and
(e) the fetus itself.
   It will be understood from the above that the sample of genomic DNA may be provided before the mother becomes pregnant, thus enabling pre-conception counseling, as well as during the pregnancy of the mother.

The PP13 gene may be analyzed by using standard methods of the art such as extracting DNA from whole blood and amplification by PCR, followed by screening by Multiphor SSCP/heteroduplex analysis or other relevant techniques.

The mutation in the PP13 gene will be expected to affect the phenotype of PP13 in the pregnant woman, i.e. it is not a silent mutation. For example, one or more of the following parameters of PP13 may be affected:
(a) level of expression of PP13 as measured in a bodily substance of the woman;
(b) accuracy of expression related to the expressed amino acid sequence, e.g. one or more amino acids may be added, deleted, repeated or substituted;
(c) post-translational processing, e.g. glycosylation, phosphorylation, methylation, etc.;
(d) trafficking, e.g. the relocation of PP13 in the placental environment, such as in endosomes;
(e) loss of function related to implantation, maternal artery modulation, binding to sugar residues in the extracellular matrix, calcium metabolism in the placenta, lysophopholipase activity, release of phospholipids or elevation of prostaglandins; and
(f) gain of function related to the above.

Examples of genomic mutations may include:
Frameshift mutations in the exons or the introns;
Any mutation in the promoter or any other regulatory elements of the LGALS13 gene that could affect its expression level, structure, processing, trafficking or activity assessed by multiple methods including the use of basic Luciperase reporter to assay transcriptional activity of promoter variants;
Any expandable repeat mutation that could impair the function of the LGALS13 or its locus on chromosome 19 or the function of the entire chromosome with respect to PP13;
Any extension in the intronic region that could affect subsequent RNA splicing or other events;
Any mutation leading to loss of heterozigocity (LOP) and changed heredity patterns;
Repeat mutations that could lead to fragile gene area or other truncated mutations;
Chromosomal mutations of the inversion, translocation or other effects;
Single nucleotide point mutations (SNIPS) at the exon or intron level;
Global regulation of the gene by way of methylation, and
Mutations related to inversion, translocation, etc.

The type of mutation can be used to determine not only whether the woman is at risk to develop preeclampsia, but also which type of preeclampsia she is at risk to develop. For example, one frameshift mutation which was found in a woman who went on to develop early onset preeclampsia is named 222delT/L74W. The DNA sequence (SEQ.ID.NO:11) of this mutant is shown in FIG. 7. In this mutation, bp #222 (=T) is deleted, thus changing amino acid #74 from L to W. This mutation results in a terminally altered and truncated PP13 molecule of 101 amino acids being produced, as shown in FIG. 8C (SEQ.ID.NO:1). Thus, the existence of a frameshift mutation of the 222delT/L74W type indicates an increased risk to develop early onset preeclampsia. In a similar manner, other mutations in the PP13 gene may be identified and correlated with specific types of preeclampsia. Also included in the invention is identification of any regulatory region of the LGALS13 gene (promoter included) that could modulate induction, expression and downstream steps leading from the DNA via RNA to a functional/dys-functional protein.

An algorithim may be developed in accordance with the method of the invention to determine the risk of the mother to develop preeclampsia on the basis of the presence of the mutations. For example, if both parents are homozygotic for a specific mutation in the PP13 gene, the fetus will also have the mutation with a probability of 100%, and the risk that the mother will develop a specific type of preeclampsia based on the identity of the mutation can be determined. In another case, if both parents are heterozygotic for a specific mutation in the PP13 gene, the fetus will have the mutation with a probability of 25%, and the risk that the mother will develop a specific type of preeclampsia based on the identity of the mutation can be determined. The algorithm may also take into consideration any specific transmission pattern, or imprinting to affect the analysis.

In another aspect of the present invention, there is provided a method for determining the risk of a pregnant woman to develop preeclampsia comprising:
(a) providing a sample of a PP13 molecule from a bodily substance of the mother;
(b) determining the structure of the PP13 molecule; and
(c) determining the risk of the woman on the basis of the structure of the PP13 molecule.

Non-limiting examples of the bodily substance include maternal blood, maternal saliva, maternal urine, amniotic fluid, umbilical cord blood, chorionic villi and placental tissue.

In one embodiment of this aspect of the invention, the PP13 molecule is the PP13 protein, and the structure of the PP13 molecule is the molecular size or amino acid sequence of the PP13 molecule. The structure of the PP13 protein may be determined, for example, immunologically or by protein chemistry, as is well known to the average skilled man of the art. For example, specific antibodies against native wild-type PP13 and/or particular mutated PP13 proteins may be used to determine the presence of native PP13 and/or mutated PP13, respectively.

In another embodiment of this aspect of the invention, the PP13 molecule is mRNA of PP13 or cDNA corresponding thereto and the structure of the PP13 molecule is the sequence of the mRNA of PP13 or cDNA corresponding thereto. The sequence of the mRNA or cDNA may be determined using standard methods of the art such as PCR amplification.

In another embodiment of this aspect of the invention, polymorphism in the LGLAS13 DNA, cDNA or RNA is used to:

a. overlay designated microchips with all types of: isoforms of SNIPS, frame shift mutations, micro-satellites or footprints or promoter activated/silenced or RNA splices or any other forms of mutations such as the ones detailed above;

b. reaction with patient tissue or bodily fluid as detailed above;

c. detection of the presence and of the nature of the mutation by way of a fluorescent, calorimetric, lanthanides or any other detection and/or amplification visual method, and

d. software of signal processing and interpretation.

As in the previous aspect of the invention, the risk of the woman to develop preeclampsia will depend on the identity of the mutated PP13 molecule. For example, if the mutated PP13 molecule is found to be the result of a frameshift mutation, this could be taken as indicating a risk to develop preeclampsia, and in certain cases, preeclampsia of a specific type. Taking the example of the 222delT/L74W mutation described above, determining that the mRNA has such a deletion could be taken as indicating a risk for developing early onset preeclampsia. As will be described below, the inventors of the present invention have succeeded in isolating the PP13-derived polypeptide encoded by the above mutated gene. Furthermore, they have shown that specific monoclonal antibodies to the native, wild-type PP13 do not recognize the mutated PP13 polypeptide. Thus, antibodies may be prepared that are specific for the mutated PP13 and may be used to identify the mutated molecule for determining the risk of developing early onset preeclampsia.

Another example of possible mutations in the PP13 molecule are mutations due to alternative splicing. The PP13 gene consists of four exons linked by three introns. In the transcription of the native PP13 molecule, the introns are excised and the exons are spliced consecutively to give the mRNA which encodes the 139 amino acid PP13 protein, as shown in FIG. 5 (SEQ.ID.NO:6). However, splicing at alternate splice sites can result in RNA spliced variants which encode PP13 proteins of lengths which differ from the native length. One example of such a mutation is the ΔEX-2 splice variant in which the majority of exon 2 and a small part of exon 3 are missing, as may be seen in FIG. 6 (SEQ.ID.NO:2). This results in a protein of 109 amino acids instead of 139 (FIGS. 6, 8B). This splice variant was obtained by screening of a cDNA library derived from a woman who went on to develop late onset preeclampsia. Thus, determining the existence of the ΔEX-2 splice variant can indicate an elevated risk to develop late onset preeclampsia.

In a further aspect of the invention, there is provided a mutated PP13 protein variant. The mutations producing the variant may be of various types and include a frameshift mutation, a point mutation, a chromosomal mutation and a mutation due to alternative splicing.

In one embodiment of this aspect of the invention, one or more of the following mutations is excluded:
(a) a −98 C/A substitution [rs3764843];
(b) IVS2-22 (A/G) [rs2233706];
(c) IVS2-36 (A/G);
(d) 222delT/L74W; and
(e) ΔEX-2.

In another embodiment of this aspect of the invention, the variant is selected from the group consisting of IVS2-36 (A/G), 222delT/L74W (SEQ.ID.NO:1) or ΔEX-2 (SEQ.ID.NO:2).

Also included in this aspect of the invention are antibodies which specifically bind to the variants, nucleic acid sequences which encode the variants and vectors comprising those sequences.

In a still further aspect of the invention, there is provided a method for purifying a mutated PP13 protein variant in soluble form comprising:
(a) expressing the PP13 variant in a host cell;
(b) disrupting the host cell and centrifuging the cell contents;
(c) re-suspending the centrifuged pellet in buffer containing a high concentration of a denaturation agent;
(d) loading the resuspended pellet on an affinity column capable of binding PP13;
(e) washing the affinity column with a gradient of the denaturation agent; and
(f) eluting the PP13 in soluble form from the column.

It is well known that expressing a foreign protein in a bacterial host often results in the expressed protein being produced in an insoluble form in “inclusion bodies”. This prevents the production of the desired protein in a useful, soluble form. This was the case also for the expression of various protein variants of PP13. The inventors have now found a method for purifying the variants so as to obtain a soluble protein. Although the method is exemplified using urea, other protein denaturation agents such as guanidine hydrochloride may be used.

Other aspects of the invention include the use of RNA tools including, but not limited to, restriction fragment length polymorphism (RFLP), short hairpin (sh)RNA or small interference (si)RNA or (mi)RNA as a means to modulate either the expression or mode of activity or process of transferability to progeny or any other way that could diversify the repertoire of PP13 on its own or in relations to preeclampsia as applied to the success of in-vitro fertilization or in-vitro diagnosis or live or in-vitro prognosis or therapy.

Further aspects of the invention include diagnostic kits for use in the methods of the invention. Such kits may include:
a. A kit for use in a method for determining the risk of a woman to develop preeclampsia comprising DNA probes for specific genomic sequences of the PP13 native gene and/or mutated sequences thereof.
b. A kit for use in a method for determining the risk of a woman to develop preeclampsia comprising antibodies against the PP13 native sequence and/or mutated sequences thereof.
c. A kit for use in a method for determining the risk of a woman to develop preeclampsia comprising RNA probes for specific sequences of the PP13 native mRNA and/or mutated sequences thereof.
d. A kit for use in a method for determining the risk of a woman to develop preeclampsia comprising DNA probes for specific sequences of the PP13 cDNA and/or mutated sequences thereof.
e. A kit for use in a method for determining the risk of a woman to develop preeclampsia comprising DNA or RNA chips comprising specific sequences of the PP13 cDNA or RNA and/or mutated sequences thereof.

In accordance with the invention, the LGALS13 gene may be characterized by one or more of the following methods of analysis:

(a) Bioinformatics
i) Promoter predictors
ii) Phylogenetic conservation/footprinting
iii) Polymorphism spectrum (SNPs, microsats, etc)

(b) Genetic mutation screening
i) Ethnic frequencies
ii) Haplotype construction and analysis
iii) Frequency in early pregnancy losses (eg, POC)—(? influence viability)

(c) Transmission studies
i) Investigate transmission pattern (Mother-to-baby)
ii) Evidence of imprinting

(d) Methylation Studies
i) Investigate global regulation of gene

(e) Characterisation of selected LGALS13 sequence variants

(f) Full genomic characterisation of templates

(g) Extend intronic regions investigated

(h) Search for LOH of exon 2, if polymorphic marker like microsatellite is available

(i) Develop allele-specific assay to “type” 222delT variant

(j) Investigate the use of PTT to identify the 222delT variant and any other potential truncating variants

(k) Investigate which methods are best suited to characterise the sequence variant [possible gene conversion event], in exon 3

(l) RNA extraction and preparation

(m) Cloning into pGEM-T Easy vector (Promega)
i) PCR and gel purification
ii) A-tailing
iii) Ligation
iv) Preparing competent cells
v) Transformation
vi) Clone selection
vii) Minprep

(n) pGL2-Basic Luciferase reporter (to assay transcriptional activity of promoter variant)
i) Vector preparation (digestion, purification, dephosphorylation, ligation)
ii) Transformation
iii) Maxiprep

(o) Constructs [in luciferase reporter vectors] representing variant and wild-type alleles will be investigated for altered transcriptional activity using FuGene 6 as transfection reagent.

(p) Cell culturing

(q) Transfected cells will be subjected to exogenous stimuli [eg, variable oestrogen levels, transient hypoxia, etc] before being harvested and analysed for luciferase activity by luminometric methods.

(r) Automated sequencing (confirmation of clones, methylation status, etc)

(s) Quantitative investigations will include the analysis of exonic variants (by the mini-gene system), while real-time PCR will be used to investigate whether the four identified intronic sequence variants influence splicing of the gene.

(t) Statistical Analysis

Other aspects of the invention will become apparent from the detailed description below.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a schematic drawing exemplifying how mutated PP13 molecules can originate from the PP13 gene;

FIG. 2 is a schematic drawing exemplifying how a PP13 protein can be prepared from a mutated cDNA by cloning and expressing in E-Coli;

FIG. 3 is a photograph of a SDS-PAGE gel showing bands made by native and mutated variants of PP13;

FIGS. 4A, 4B and 4C are plots of antibody binding as a function of antibody dilution for the native and mutated variants of PP13, using specific anti-PP13 antibodies (4A and 4B) or control anti-histidine antibody (4C);

FIG. 5 shows the DNA (SEQ.ID.NOS:3&4) and amino acid (SEQ.ID.NOS:5-8) sequences of native recombinant PP13 (rPP13);

FIG. 6 shows the DNA (SEQ.ID.NOS:9&10) and amino acid (SEQ.ID.NO:2) sequences of the ΔEX-2 PP13 splice variant;

FIG. 7 shows the DNA sequence (SEQ.ID.NO:11) of the 222delT/L74W mutation. The T deletion occurrs between the underlined nucleotides;

FIGS. 8A, 8B and 8C show the amino acid sequences of the native rPP13 (SEQ.ID.NO:6), the ΔEX-2 PP13 variant (SEQ.ID.NO:2) and the 222delT/L74W variant (SEQ.ID.NO:1), respectively. The deletion in the latter varient causes a frameshift which creates 28 new amino acids in the terminal region (underlined) up to a stop codon (#); and

FIGS. 9A, 9B, 9C, 9D and 9E show a polymorphism analysis of a South African cohort of primigravida consisting of 80 cases of early (<34 weeks of gestation age (GA)) preeclamsia and ˜100 controls. (9A) polymorphism frequency analysis; (9B) gel of the exon 3.1 SSCP/hetroduplex; (9C) delT222/-electropherogram; (9D) delT alignment with wild type; and (9E) the relative positions of the different mutants with respect to the wild type gene. 3 new Ser; 3 Cys deleted & 2 novel; E⁷⁵ CHO binding site removed.

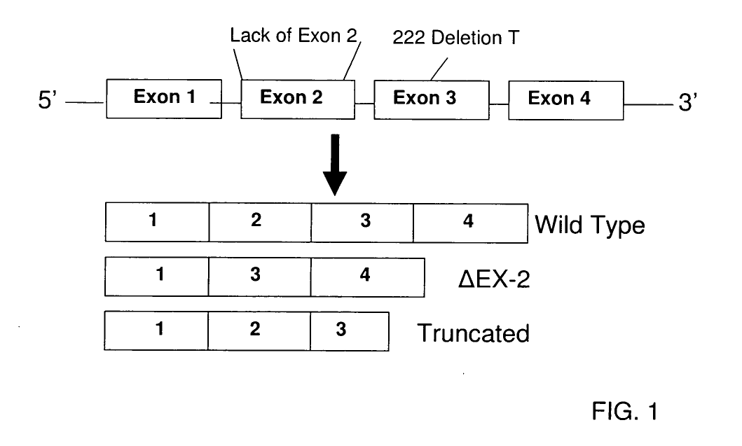

### DETAILED DESCRIPTION OF EXEMPLARY EMBODIMENTS

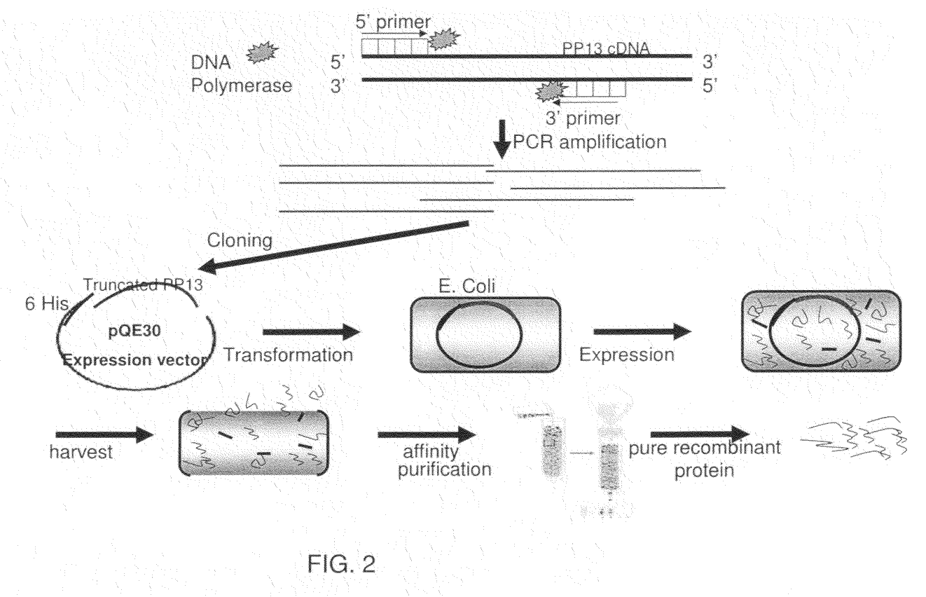

1. Cloning, Expression and Purification of 222delT/L74W and ΔEX-2 PP13 Variants (FIG. 2)

A. Polymerase Chain Reaction (PCR) for the 222de1T/L74W Mutation Variant

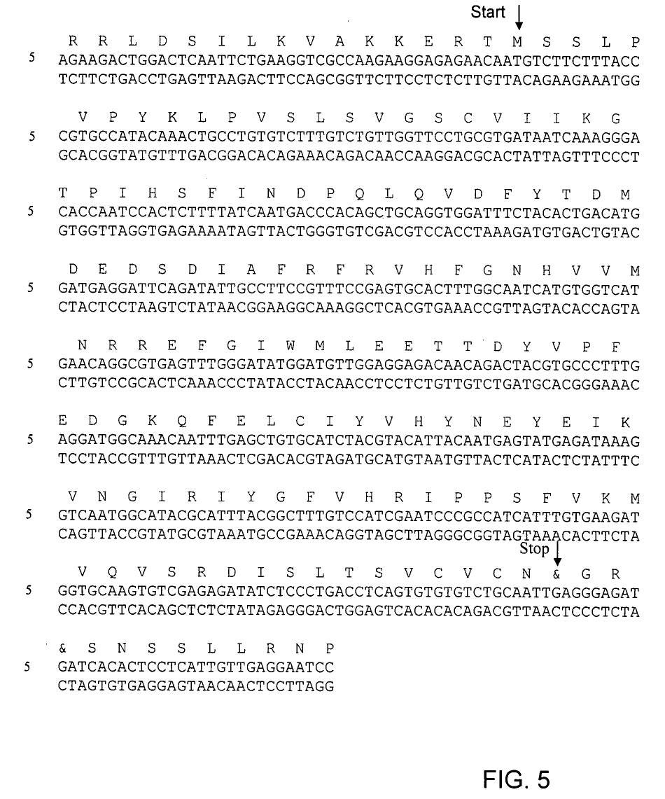

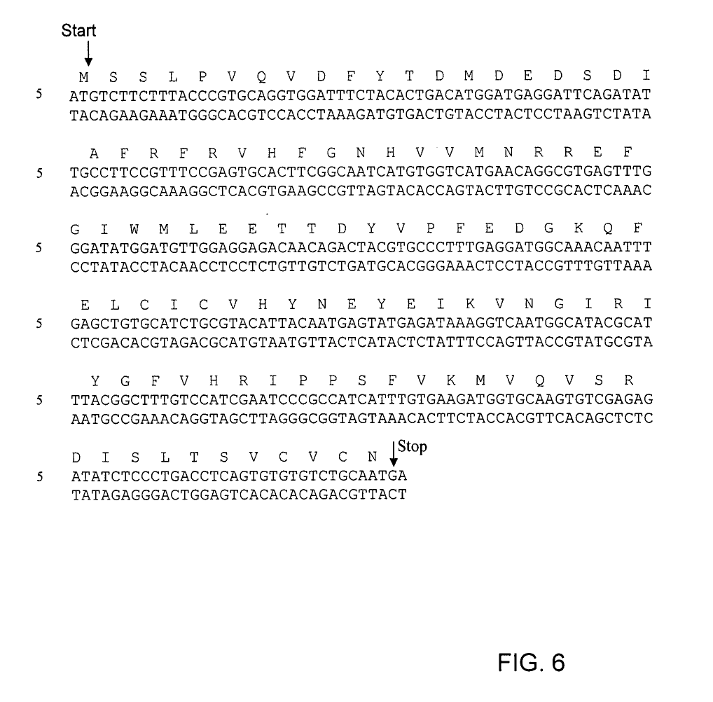

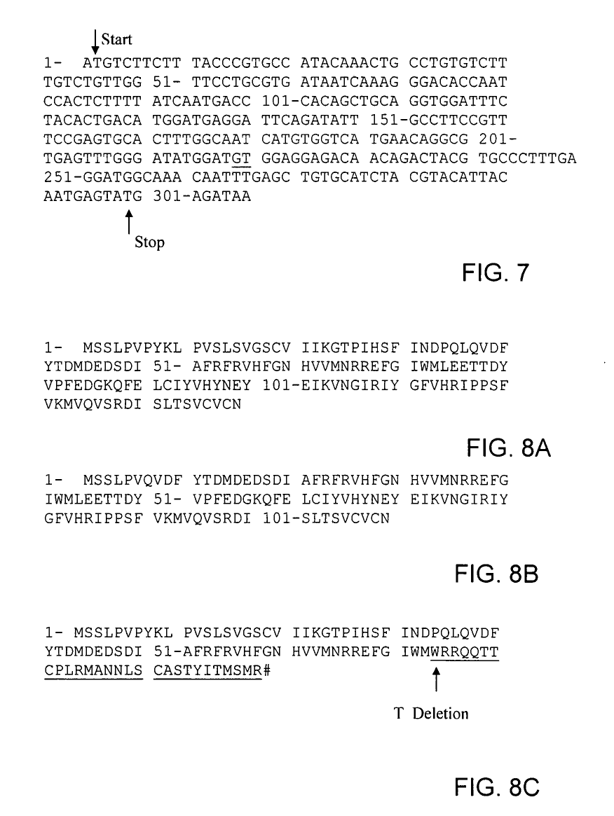

Based on a polymorphism analysis of the PP13 gene and correlation with the occurrence of polymorphism and the development of preeclampsia, the sequence of PP13 wild type (FIG. 5) was used as a template to generate the 222delT/L74W (also referred to herein as the truncated) sequence by PCR techniques. Two primers were designed with the following sequences: a sense primer: CGAATCCATGTCTTCTTTACCCGTGC (SEQ.ID.NO:12) and an anti-sense primer:

(SEQ. ID. NO: 13)

TAAGTCGAGCTCCATCCATATCCCAAACTCAC.

The restriction site sequences of BamH I and Sac I were introduced in the sense and anti-sense primers respectively. Both primers were synthesized by Sigma-Genosys.

To amplify the truncated PP13 DNA sequence, 1 ng of wild type PP13 DNA (in plasmid) was used as a template. 0.1-1 μM of the above mentioned specific primers, 1 U of Pfu DNA polymerase (Promega), 200 μM dNTP-mix and Pfu DNA polymerase ×10 buffer. PCR was carried out at the following high temperature cycles: 94° C. for 2 min, 94° C. for 30 sec, 60° C. for 30 sec and 72° C. for 1 min over 35 cycles. A final extension was carried out at 72° C. for 4 min and the PCR product, analyzed by agarose gel and revealing the expected size of 288 bp, was stored at 4° C. until use.

B. PCR for the ΔEX-2 Variant

Screening of a cDNA library derived from a preeclamptic placenta revealed an exon-2 deleted (also referred to herein as the spliced) sequence (deletion of 30 amino acids) of PP13. Based on nucleotide sequence analysis of the deleted PP13 variant, a set of primers was designed to flank the full length of the DNA. Two primers were designed with the following sequences: a sense primer: 5′-CGATACGGATCCATGTCTTCTTTACCCGTGC-3′ (SEQ.ID.NO:14) and an anti-sense primer: 5′-TAAGTCGAGCTCATTGCAGACACACACTGAGG-3′ (SEQ.ID.NO:15). Both primers were synthesized by Sigma-Genosys.

To amplify the deleted ΔEX-2 PP13 DNA sequence, 1 ng of deleted PP13 DNA was used as a template, 0.1-1 μM of the above mentioned specific primers, 1 U of Pfu DNA polymerase (Promega), 200 μM dNTP-mix and Pfu DNA polymerase ×10 buffer. PCR was carried out at the following high temperature cycles: 94° C. for 2 min, 94° C. for 30 sec, 55° C. for 30 sec and 72° C. for 1 min over 35 cycles. A final extension step was carried out at 72° C. for 4 min and the PCR product, analyzed by agarose gel and revealing the expected size of 338 bp, was stored at −20° C. until use. The resulting PCR fragments were inserted into a pUC57-T cloning vector (T-Cloning Kit #1212MBI Fermentase) and the clones containing the insert were selected and sequenced by automated DNA sequencing at the Biological Services at the Weizmann Institute, Rehovot, Israel.

2-Cloning of the Truncated and Spliced form DNA into Expression Vectors.

A-Ligation: The PCR products of the truncated and spliced PP13 DNA were purified using a QIAquick PCR purification kit prior to ligation. The Purified PP13 DNA product (1 μg) and the expression vector pQE 30 (0.5 μg, Qiagen) were digested with BamH I and Sac I (20 U each, New England Biolabs-NEB) in NEBuffer BamH I and NEBuffer Sac I, respectively. Insert:vector ratios of 3:1, 1:1 and 1:3 were used for ligation of the digested PCR product DNA with 50 ng of digested pQE-30 using 100 U of T4 ligase (NEB) and T4 ligase buffer for 2 hr at 22° C.

B-Transformation: The ligation mixture was transformed into M15 (pREP4) cells (Qiagen) and 10 μl of the ligation mixture were added to 100 μl Competent M15 pREP4) cells for 10 min in ice and then transferred to a 42° C. water bath for 50 sec. After heat shock, the mixture was placed on ice for another 2 min and 900 μl of LB medium was added to the transformation reaction and incubated for 60 min at 37° C. with shaking of approximately 225 rpm. 10-100 μl of the cells were plated on LB agar plate containing 100 μg/ml ampicillin (Sigma) and 25 μg/ml Kanamycin (Sigma) for overnight at 37° C.

C-Screening for positive colonies: 20 single colonies grown on the plate were picked and cultured in 2 ml LB medium containing ampicillin (100 μg/ml) and kanamycin (μg/ml) for overnight at 37° C. with 225 rpm shaking. Plasmid DNA was purified from each colony culture with Wizard Plus SV minipreps DNA purification system (Promega). The presence of the PP13 DNA insert was tested by PCR as follow: the PCR reaction (20 μl volume) composed of 1 ng of DNA template, 0.1-1 μM truncated PP13 and spliced variant specific respective primers and 10 ml of ×2 ready mix for PCR (Bio-Lab Ltd). The PCR conditions were as detailed above. PCR products were separated on 1.5% agarose and the DNA bands were visualized in LAS-3000 image system (Fuji). The potential positive clones (4) were selected according to the calculated size of the PCR product. The final DNA sequence of each clone was determined by sequencing carried out in the multi-disciplinary laboratories unit (Rappaport Institute of Medical Science—Technion, Haifa).

### 3-Expression of the Truncated and Spliced PP13

Based on verified sequence analyses, one positive clone was selected for expression of the protein and inoculated in 20 ml of LB medium containing ampicillin and Kanamycin at 37° C. for overnight with shaking. The culture was mixed 1:50 in LB medium containing antibiotics and grown at 37° C. until reaching an OD600 of 0.6. The expression of the protein was induced with 1 mM—isopropyl-b-D-thiogalactopyranoside-IPTG for 3 hrs. Bacterial cells were harvested by centriftigation at 4000 g×20 min at 4° C. The cell pellet was stored until use at −80° C. Aliquots were tested by SDS-PAGE analysis to determine the molecular weight of the recombinant protein.

### 4. Purification of Truncated and Spliced PP13

Based on SDS-PAGE analysis, the recombinant, truncated PP13 was localized to be trapped in the inclusion bodies. The method used to obtain soluble polypeptides was as follows.

Cell pellet was resuspended in lysis buffer containing 20 mM Tris-HCl, pH 8, 150 mM NaCl, 5 mM Imidazole and protease inhibitor (Roche), 10% glycerol and incubated with 0.2 mg/ml lysozyme (Sigma) for 1 hr at 4° C. The cells were disrupted by sonication on ice 6×10 sec of 200 W or alternatively disrupted by applying pressure of 1000 PSi in minicell French press (Thermo). Soluble proteins were discarded and the pellet containing the inclusion bodies (0.75 gr) was resuspended in binding buffer (20 mM Tris-HCl, pH 8.0, 300 mM NaCl, 5 mM Imidazole, 6 M Urea, PMSF, Complete (protease inhibitor—Roche), 1 mM DTT and 10% glycerol). After 1 hr of incubation at room temperature, the insoluble proteins were discarded by centrifugation at 20,000 g for 20 min (SS34 rotor, Sorval-RC). The soluble fraction was filtered through 0.45 μm pore size filters and mixed with 1 ml of pre-equilibrated Ni-NTA agarose (Qiagen) for 1 hr at RT. The refolding of the bound recombinant truncated PP13 was performed on the column using a step-wise linear 6-0 M urea gradient. First the Ni-NTA agarose column was washed with 10 ml of wash buffer (20 mM Tris-HCl, pH 8.0, 300 mM NaCl, 20 mM Imidazole, 6 M Urea, PMSF, Complete, 1 mM DTT and 10% glycerol) followed by washing the column with 10 ml of refolding buffers (wash buffer containing 4, 2, 1, 0.5 and 0 M urea). Bound recombinant PP13 was eluted with 5 ml of elution buffer (20 mM Tris-HCl, pH 8.0, 300 mM NaCl, 0.5 M Imidazole, PMSF, Complete, 1 mM DTT and 10% glycerol). Recombinant, truncated PP13 protein was dialyzed against TBS (20 mM Tris-HCl, pH-8, 150 mM NaCl) and diluted with equal volume of 60% glycerol in TBS and stored at −80° C. until use. The protein concentration was determined by Bradford assay and stored at −20° C. for further analysis.

### 5. Characterization of the Recombinant Truncated and Spliced PP13 Variants

### A. SDS-PAGE

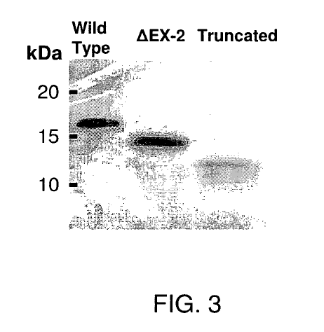

Recombinant truncated and spliced PP13 variants (1-5 μg) were resuspended in sample buffer in the presence of 5% β-mercaptoethanol and boiled for 5 min at 95° C. Proteins were loaded on 15% SDS-PAGE and separated by applying 120 volts for approximately 2 hrs. To visualize the protein bands, the gel was washed with H₂O for min and stained for 1 hr with GelCode reagent (Pierce). The staining reagent traces were removed by several washes with H₂O until reaching enough clarity of the stained PP13. The approximate molecular size of the PP13 protein variants was determined by molecular weight standard proteins which were separated in parallel on the same gel and compared to the calculated molecular size of the protein based on its amino-acid composition. The gel is shown in FIG. 3.

It may be seen that the native, wild-type PP13 has the highest molecular size (appx. 16-17 kDa), followed by the spliced (appx. 14-15 kDa) and the truncated (appx. 12-13 kDa) variants.

### B. Enzyme Linked Immuno-Assay (ELISA)

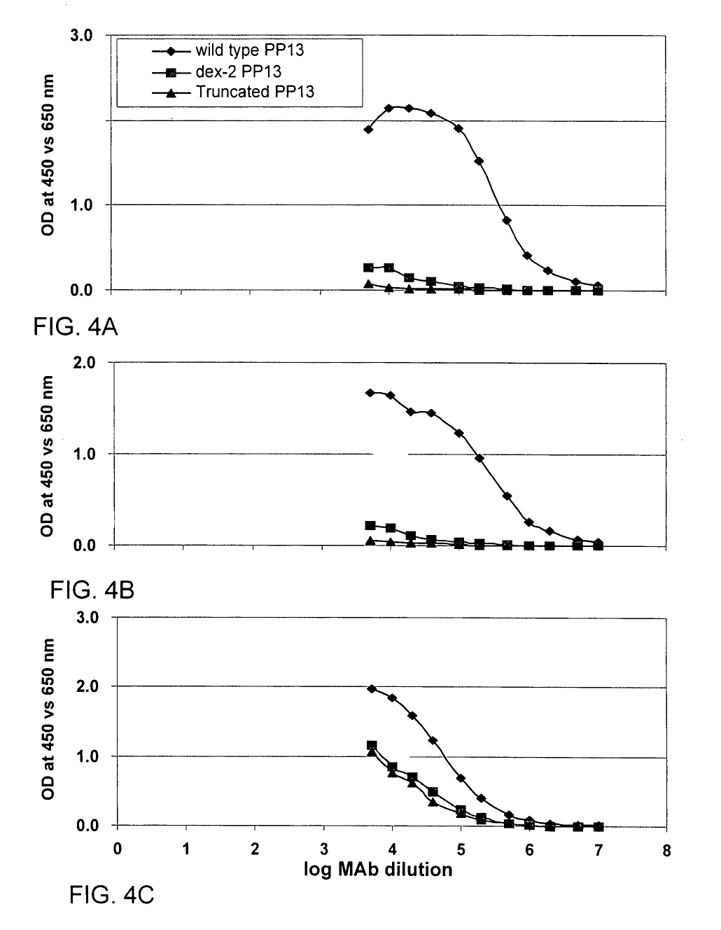

The ELISA test was used to test the recognition of the truncated recombinant PP13 by anti-PP13 monoclonal antibodies. Briefly, micro-plate wells were coated with 1-10 μg/ml of the recombinant wild-type, truncated and spliced PP13s for 2 hrs at 37° C. followed by blocking the free binding sites by 1% Bovine serum albumin-BSA in Carbonate buffer for 1 hr. Coated proteins were incubated with serial dilution of the following monoclonal antibodies: clones 27-2-3, 215-28-3 and 534-16 and anti-Histidine (control) for overnight at 4° C. Unbound antibodies were washed with phosphate buffer saline containing 0.05% Tween-20. Goat anti-mouse IgG conjugated to HRP was used for detecting bound antibodies followed and TMB was used a substrate for the HRP. The optical density of the resulting enzymatic product was measured with an ELISA reader at 650 nm. The reaction was stopped after 30 min and the optical density was re-measured at 450 vs. 650 nm. The results are shown in FIGS. 4A, 4B and 4C.

It may be seen that while the wild type PP13 reacted with the specific anti-PP13 antibodies, the truncated and spliced variants did not (FIGS. 4A & 4B). All of the PP13 proteins reacted with the control anti-histidine antibody (FIG. 4C). This may provide an explanation for the observation that during the first trimester, a woman with high risk for preclampsia has a low measured amount of PP13 in her bodily substances.

### C. Dot Blot Analysis

Wild type, truncated and spliced PP13s were absorbed to nitrocellulose membrane (Biorad) and free binding sites were blocked with 5% milk in Tris buffer Saline pH 8.0 (TBS) for 1 hr. Membrane was incubated with anti PP13 monoclonal antibodies (clones 27-2-3, 215-28-3 and 534-16) for 2 hrs at 37° C. and free antibodies were washed with TBS-tween 20. To detect bound anti-PP13 antibodies, a secondary antibody of goat anti-mouse IgG conjugated to HRP enzyme was added to the membrane and incubated for 90 min at room temperature (RT) followed by discarding the free excess antibodies by washes as indicated above. Enhanced Chemiluminescene (ECL) reagents were used as a substrate for the HRP and the signals were visualized, captured and analyzed by using the LAS3000 image system (Fuji).

D. The 222deltT/L74W Mutation

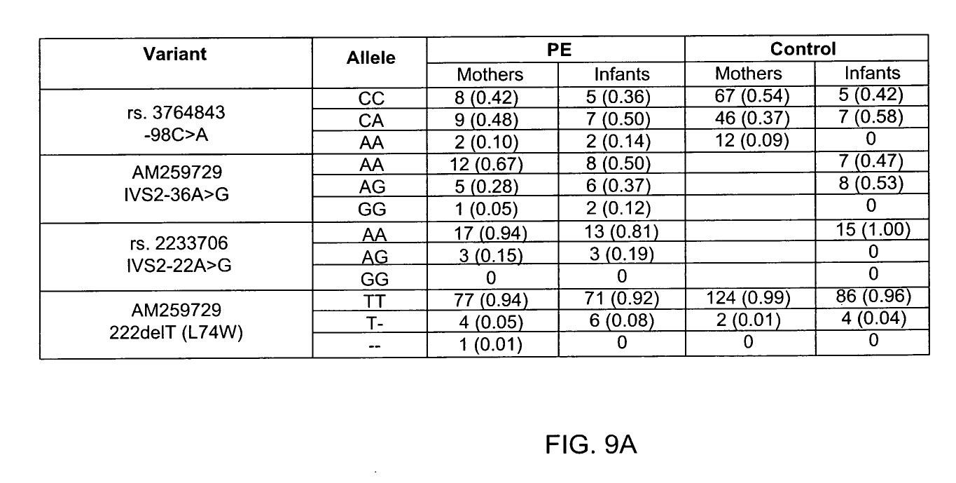

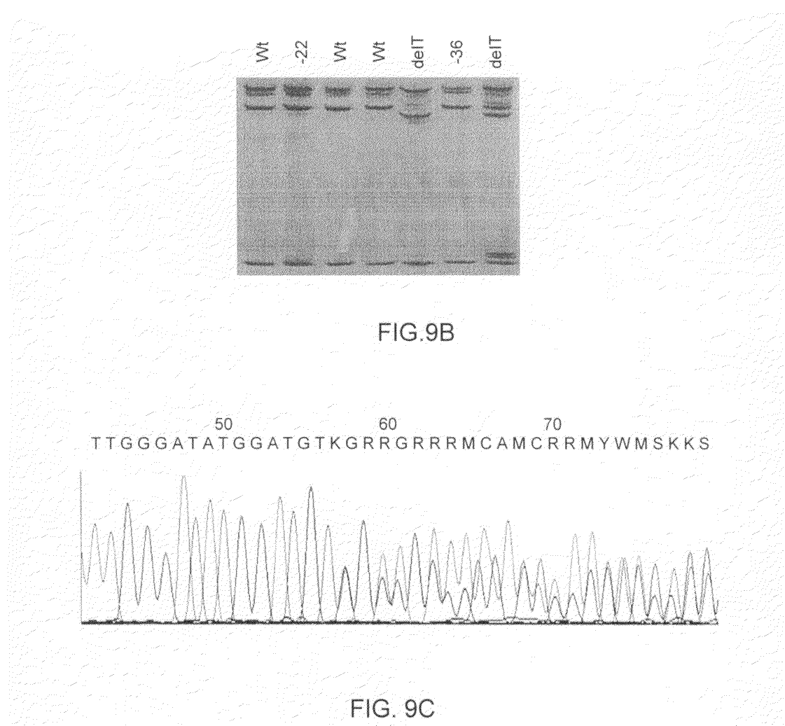

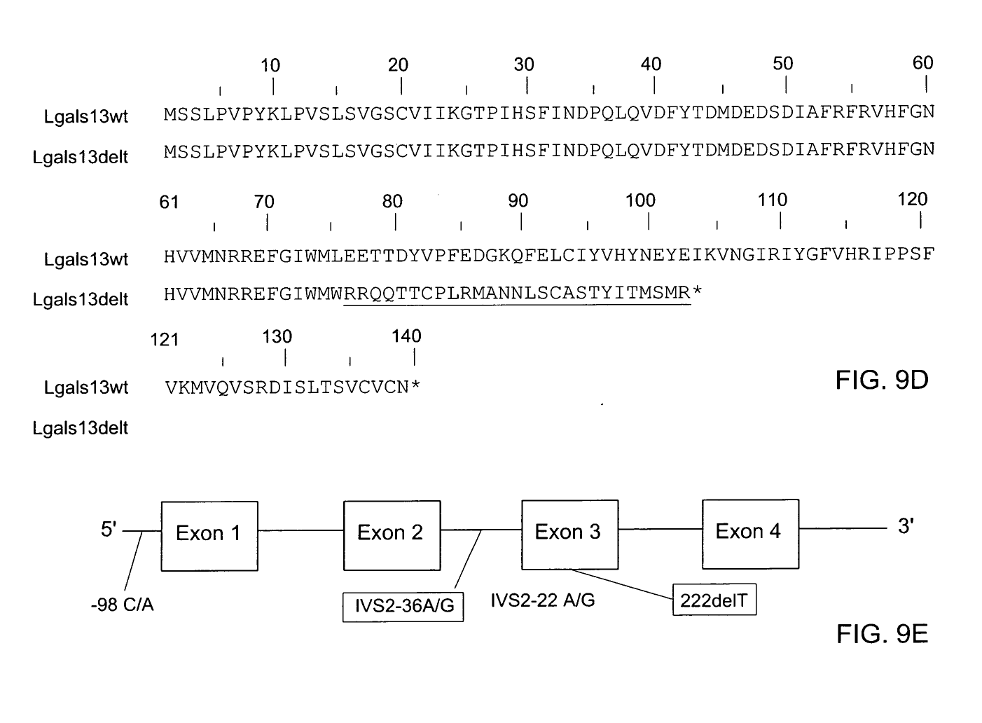

The following is a description of the identification and analysis of the 222deltT/L74W mutation, with reference to FIGS. 9A-9E.

Intronic oligonucleotide primer sets for PCR were designed to flank each of the four LGALS-13 gene exons as well as a short portion of the 5′ and 3′ untranslated regions. Each generated amplicon was subjected to Multiphor SSCP/heteroduplex analysis (FIG. 9B). Conformational variants were further characterized by automated sequencing and where appropriate, by restriction enzyme analysis. The intronic variants were genotyped in a small group of primigravida patients (n<20).

The identified deletion was further characterized in a larger cohort (n>80) of primigravida patient who developed early (GA<34 weeks) preeclampsia, their infants and a matched control group of ˜100 individuals, comprising healthy mothers and unrelated newborn infants. The results of the analysis are summarized in the table below (FIG. 9A).

TABLE 1
Polymorphism analysis of South African cohort of primigravida

| | | PE | | Control | |
|---|---|---|---|---|---|
| Variant | Allele | Mothers | Infants | Mothers | Infants |
| rs, 3764843- | CC | 8 (0.42) | 5 (0.36) | 67 (0.54) | 5 (0.42) |
| 98C>A | CA | 9 (0.48) | 7 (0.50) | 46 (0.37) | 7 (0.58) |
| | AA | 2 (0.10) | 2 (0.14) | 12 (0.09) | 0 |
| AM259729 | AA | 12 (0.67) | 8 (0.50) | | 7 (0.47) |
| IVS2-36A>G | AG | 5 (0.28) | 6 (0.37) | | 8 (0.53) |
| | GG | 1 (0.05) | 2 (0.12) | | 0 |
| rs. 2233706 | AA | 17 (0.85) | 12 (0.81) | | 15 (1.00) |
| IVS-22A->G | AG | 3 (0.15) | 3 (0.19) | | 0 |
| | GG | 0 | 0 | | 0 |
| AM259729 | TT | 77 (0.94) | 71 (0.92) | 124 (0.99) | 86 (0.96) |
| 222deIT | T— | 4 (0.05) | 6 (0.08) | 2 (0.01) | 4 (0.04) |
| (L74W) | — — | 1 (0.01) | 0 | 0 | 0 |

Four sequence variants were identified in this cohort. The majority of the preeclamptic patients carried 1-2 mutations in the PP13 gene. Among them, the 222deltT/L74W mutation associated with truncated PP13 was discovered in 6% preeclamptic (5% hetro and 1% homozygotes, compared to 1% among control), and 8% of their infants (compared to 4% in the control) inferring a higher proportion among the early (GA<34 weeks) preeclampsia vs. control and an inferred paternal contribution to the route of transfer to the newborn.

Point mutations between Exons 2 and 3, that could be critical for the development of spliced variants in Exon 2 (Mutation IVS2-22A>G and IVS2-36A>G)) appear only in the preeclamptic cases (15% and 28% respectively) and only in a heterozygous form, and are also detected at a little higher frequency in the newborns (19% and 37% respectively) The deletion (T) frame-shift mutation (222deltT/L74W) was detected in exon-3 in preeclamptic patients, their infants and paternal contribution was inferred in several cases. The mutation is predicted to create a novel 28 or 27 C terminal region which is 38 or 37 amino acids shorter than the wild-type PP13.

The mutant exon 3.1 SSCP/hetroduplex was run in a gel against the wild type (FIG. 9B). The mutant delT222/—was analyzed using an electropherogram (FIG. 9C). An alignment of the amino acid sequences of Lgals13 wt and Lgals13delT is shown in FIG. 9D. The delT frameshift creates a new 27AA terminal region (underlined) which is 37AA shorter than the wild type peptide. FIG. 9E shows the relative positions of the different mutants with respect to the wild type gene.

1. A method for determining the risk of a mother of a fetus to develop preeclampsia during pregnancy comprising:
(a) providing a sample of genomic DNA from an individual related to the fetus;
(b) analyzing the DNA for the presence of one or more mutations in the PP13 gene; and
(c) determining the risk of the mother on the basis of the presence of the mutations.

2. The method of claim 1 wherein the individual related to the fetus is selected from the following:
(a) the mother of the fetus, or her mother or father;
(b) the father of the fetus or his mother or father;
(c) a sibling of the fetus or siblings of the father or of the mother of the fetus;
(d) the placenta of the pregnancy or of a twin pregnancy or the placenta from previous pregnancies of the mother; and
(e) the fetus.

3. The method of claim 1 wherein the mutation in the PP13 gene affects one or more of the following parameters of PP13:
(a) level of expression;
(b) amino acid sequence;
(c) post-translational processing;
(d) trafficking;
(e) loss of function; and
(f) gain of function.

4. The method of claim 1 wherein the mutation in the PP13 gene is selected from the following:
(a) frameshift mutations;
(b) chromosomal mutations;
(c) point mutations; and
(d) RNA splicing.

5. The method of claim 1 wherein in step (c) the presence of a frameshift mutation indicates a risk to develop early onset preeclampsia.

6. The method of claim 5 wherein the frameshift mutation is 222delT/L74W.

7. The method of claim 1 wherein the sample of genomic DNA is provided before the mother becomes pregnant.

8. The method of claim 1 wherein the sample of genomic DNA is provided during the pregnancy of the mother.

9. A method for determining the risk of a pregnant woman to develop preeclampsia comprising:
(a) providing a sample of a PP13 molecule from a bodily substance of the mother;
(b) determining the structure of the PP13 molecule; and
(c) determining the risk of the woman on the basis of the structure of the PP13 molecule.

10. The method of claim 9 wherein the bodily substance is selected from maternal blood, maternal saliva, maternal urine, amniotic fluid, umbilical cord blood, chorionic villi and placental tissue.

11. The method of claim 9 wherein the PP13 molecule is PP13 protein, and the structure of the PP13 molecule is the molecular size or amino acid sequence of the PP13 molecule.

12. The method of claim 11 wherein the structure of the PP13 is determined immunologically or by protein chemistry.

13. The method of claim 12 wherein the immunological determination is made by use of specific antibodies against PP13 and/or mutated PP13.

14. The method of claim 9 wherein the PP13 molecule is mRNA of PP13 or cDNA corresponding thereto and the structure of the PP13 molecule is the sequence of the mRNA of PP13 or cDNA corresponding thereto.

15. The method of claim 9 wherein in step (c), the presence of a truncated PP13 due to a frameshift mutation indicates a risk to develop early onset preeclampsia.

16. The method of claim 15 wherein the frameshift mutation is 222delT/L74W.

17. The method of claim 9 wherein in step (c), the presence of a splice variant PP13 due to alternative splicing indicates a risk to develop late onset preeclampsia.

18. The method of claim 17 wherein the splice variant is ΔEX-2.

19. A mutated PP13 protein variant.

20. The PP13 variant of claim 19 wherein the mutation is selected from the following:
(a) a frameshift mutation;
(b) a point mutation; and
(c) a mutation due to alternative splicing.

21. The PP13 variant of claim 20 with the proviso that one or more of the following mutations is excluded:
(a) a C/A substitution [rs3764843];
(b) IVS2-22 (A/G) [rs2233706];
(c) IVS2-36 (A/G);
(d) 222delT/L74W; and
(e) ΔEX-2.

22. The PP13 variant of claim 20 wherein the variant is selected from the group consisting of IVS2-36 (A/G), 222delT/L74W (SEQ.ID.NO:1) or ΔEX-2 (SEQ.ID.NO:2).

23. A method for purifying a mutated PP13 protein variant in soluble form comprising:
(a) expressing the PP13 variant in a host cell;
(b) disrupting the host cell and centrifuging the cell contents;
(c) resuspending the centrifuged pellet in buffer containing a high concentration of a denaturation agent;
(d) loading the resuspended pellet on an affinity column capable of binding PP13;
(e) washing the affinity column with a gradient of the denaturation agent; and
(f) eluting the PP13 in soluble form from the column.

24. The method of claim 23 wherein the denaturation agent is urea.

25. A kit for use in a method for determining the risk of a woman to develop preeclampsia comprising DNA probes for specific genomic sequences of the PP.13 native gene and/or mutated sequences thereof.

26. A kit for use in a method for determining the risk of a woman to develop preeclampsia comprising antibodies against the PP13 native sequence and/or mutated sequences thereof.

27. A kit for use in a method for determining the risk of a woman to develop preeclampsia comprising RNA probes for specific sequences of the PP13 native mRNA and/or mutated sequences thereof.

28. A kit for use in a method for determining the risk of a woman to develop preeclampsia comprising DNA probes for specific sequences of the PP13 cDNA and/or mutated sequences thereof.

29. A kit for use in a method for determining the risk of a woman to develop preeclampsia comprising DNA or RNA chips comprising specific sequences of the PP13 cDNA or RNA and/or mutated sequences thereof.
